(19) United States
(12) Patent Application Publication
(10) Pub. No.: US 2026/0162762 A1
(43) Pub. Date: Jun. 11, 2026

(54) COMPLEX STRUCTURE DETERMINATION METHOD AND APPARATUS, DEVICE, STORAGE MEDIUM, AND PROGRAM PRODUCT

(71) Applicant: TENCENT TECHNOLOGY (SHENZHEN) COMPANY LIMITED, Shenzhen (CN)

(72) Inventors: Fandi WU, Shenzhen (CN); Jiaxiang WU, Shenzhen (CN)

(21) Appl. No.: 19/182,003

(22) Filed: Apr. 17, 2025

Related U.S. Application Data
(63) Continuation of application No. PCT/CN2024/077875, filed on Feb. 21, 2024.

(30) Foreign Application Priority Data
Apr. 14, 2023 (CN) 202310437741.5

(51) Int. Cl. G16B 15/20; G16B 15/30; G16B 30/10; G16B 40/20

(52) U.S. Cl. CPC G16B 15/20; G16B 15/30; G16B 30/10; G16B 40/20

(57) ABSTRACT

A determination method includes obtaining an amino acid sequence pair of a first protein and an amino acid sequence of a second protein, generating an amino acid code of the first protein, a first amino acid pair code of the first protein, and a first protein structure of the first protein based on the amino acid sequence pair of the first protein, generating a multiple sequence alignment (MSA) code of the second protein, a second amino acid pair code of the second protein, and a second protein structure of the second protein based on the amino acid sequence of the second protein, and generating a structure of a complex of the first protein and the second protein based on the amino acid code, the first amino acid pair code, the first protein structure, the MSA code, the second amino acid pair code, and the second protein structure.

### CROSS-REFERENCE TO RELATED APPLICATIONS

This application is a continuation of International Application No. PCT/CN2024/077875, filed on Feb. 21, 2024, which claims priority to Chinese Patent Application No. 2023104377415, entitled “COMPLEX STRUCTURE DETERMINATION METHOD AND APPARATUS, DEVICE, STORAGE MEDIUM, AND PROGRAM PRODUCT” and filed on Apr. 14, 2023, the entire contents of both of which are incorporated herein by reference.

### FIELD OF THE TECHNOLOGY

This application relates to the technical field of artificial intelligence, and in particular, to a complex structure determination method and apparatus, a device, a storage medium, and a program product.

### BACKGROUND OF THE DISCLOSURE

In medical-related fields, determination of a structure of a complex formed by a plurality of proteins is an important means in fields of medicine research and development, auxiliary diagnosis, and the like.

Determination of a structure of a complex of an antigen and an antibody is taken as an example. In the related art, for each amino acid sequence in the antigen-antibody complex, a sequence library is searched separately to construct multiple sequence alignment (MSA), MSA information is matched, and the structure of the complex is predicted by using an artificial intelligence (AI) model based on a matching result.

However, one antigen corresponds to a variety of antibodies, and according to the solution in the related art, MSA information of an amino acid sequence of each type of antibody is needed, resulting in time-consuming sequence library searching and MSA construction, and low efficiency of determination of a complex structure.

### SUMMARY

In accordance with the disclosure, there is provided a determination method including obtaining an amino acid sequence pair of a first protein and an amino acid sequence of a second protein, generating an amino acid code of the first protein, a first amino acid pair code of the first protein, and a first protein structure of the first protein based on the amino acid sequence pair of the first protein, generating a multiple sequence alignment (MSA) code of the second protein, a second amino acid pair code of the second protein, and a second protein structure of the second protein based on the amino acid sequence of the second protein, and generating a structure of a complex of the first protein and the second protein based on the amino acid code, the first amino acid pair code, the first protein structure, the MSA code, the second amino acid pair code, and the second protein structure.

Also in accordance with the disclosure, there is provided a computer device including a processor, and a memory storing at least one computer program that, when executed by the processor, causes the processor to obtain an amino acid sequence pair of a first protein and an amino acid sequence of a second protein, generate an amino acid code of the first protein, a first amino acid pair code of the first protein, and a first protein structure of the first protein based on the amino acid sequence pair of the first protein, generate a multiple sequence alignment (MSA) code of the second protein, a second amino acid pair code of the second protein, and a second protein structure of the second protein based on the amino acid sequence of the second protein, and generate a structure of a complex of the first protein and the second protein based on the amino acid code, the first amino acid pair code, the first protein structure, the MSA code, the second amino acid pair code, and the second protein structure.

Also in accordance with the disclosure, there is provided a non-transitory computer-readable storage medium storing at least one computer program that, when executed by a processor, causes the processor to obtain an amino acid sequence pair of a first protein and an amino acid sequence of a second protein, generate an amino acid code of the first protein, a first amino acid pair code of the first protein, and a first protein structure of the first protein based on the amino acid sequence pair of the first protein, generate a multiple sequence alignment (MSA) code of the second protein, a second amino acid pair code of the second protein, and a second protein structure of the second protein based on the amino acid sequence of the second protein, and generate a structure of a complex of the first protein and the second protein based on the amino acid code, the first amino acid pair code, the first protein structure, the MSA code, the second amino acid pair code, and the second protein structure.

### BRIEF DESCRIPTION OF THE DRAWINGS

To describe the technical solutions in the embodiments of this application or in the related art more clearly, the following briefly introduces the accompanying drawings required for describing the embodiments or the related art. Apparently, the accompanying drawings in the following description show only some embodiments of this application, and those of ordinary skill in the art may still derive other drawings from these accompanying drawings without creative efforts.

FIG. 1 is a schematic diagram of a system used in a complex structure determination method according to this application.

FIG. 2 is a flowchart of a complex structure determination method according to an exemplary embodiment of this application.

FIG. 3 is a schematic diagram showing a user interface according to this application.

FIG. 4 is a flowchart of a complex structure determination method according to an exemplary embodiment of this application.

FIG. 5 is a block diagram of a complex structure prediction model according to an embodiment of this application.

FIG. 6 is a flowchart of a model training method according to an exemplary embodiment of this application.

FIG. 7 is a schematic structural diagram showing a model according to this application.

FIG. 8 is a schematic structural diagram showing an antibody structure prediction module according to this application.

FIG. 9 is a schematic diagram showing a process of generating an amino acid feature according to this application.

FIG. 10 is a schematic diagram showing a process of generating an amino acid pair feature according to this application.

FIG. 11 is a block diagram of a complex structure determination apparatus according to an embodiment of this application.

FIG. 12 is a structural block diagram of a computer device according to an exemplary embodiment of this application.

### DESCRIPTION OF EMBODIMENTS

The technical solutions in the embodiments of this application are clearly and completely described below with reference to the accompanying drawings in the embodiments of this application. Apparently, the described embodiments are merely some rather than all of the embodiments of this application. All other embodiments obtained by those of ordinary skill in the art based on the embodiments of this application without creative efforts fall within the scope of protection of this application.

“A plurality of” mentioned herein means two or more. “And/or” describes an association relationship between associated objects and represents that three relationships may exist. For example, A and/or B may represent the following three cases: only A exists, both A and B exist, and only B exists. The mark “/” generally indicates an “or” relationship between the associated objects.

The embodiments of this application provide a complex structure determination method for protein binding, which can improve efficiency of determining a structure of a complex. For convenience of understanding, some terms involved in this application are simply described below.

### 1) Artificial Intelligence (AI)

AI involves a theory, a method, a technology, and an application system that use a digital computer or a machine controlled by the digital computer to simulate, extend, and expand human intelligence, perceive an environment, obtain knowledge, and use knowledge to obtain an optimal result. In other words, AI is a comprehensive technology in computer science and attempts to understand the essence of intelligence and produce a new intelligent machine that can react in a manner similar to human intelligence. AI is to study the design principles and implementation methods of various intelligent machines, to enable the machines to have the functions of perception, reasoning, and decision-making.

The AI technology is a comprehensive discipline that spans a wide range of fields, including both hardware-level technologies and software-level technologies. The basic AI technologies generally include technologies such as a sensor, a dedicated AI chip, cloud computing, distributed storage, a big data processing technology, an operating/interaction system, and electromechanical integration. AI software technologies mainly include several major directions such as a computer vision technology, a speech processing technology, a natural language processing technology, and machine learning (ML)/deep learning. A display device including an image obtaining component shown in this application mainly relates to the computer vision technology and directions such as ML/deep learning, autonomous driving, and intelligent transportation.

### 2) ML

ML is a multi-field interdiscipline, and relates to a plurality of disciplines such as the probability theory, statistics, the approximation theory, convex analysis, and the algorithm complexity theory. ML specializes in studying how a computer simulates or implements a human learning behavior to obtain new knowledge or skills, and reorganize an existing knowledge structure, so as to keep improving its performance. ML is the core of AI, is a basic way to make the computer intelligent, and is applied to various fields of AI. ML and deep learning generally include technologies such as an artificial neural network, a belief network, reinforcement learning, transfer learning, inductive learning, and learning from demonstrations.

### 3) Antibody

An anti-body is a large Y-shaped protein, which is excreted by a blood cell (an effector B cell) and is used by the immune system to recognize and neutralize a foreign substance such as a bacteria or a virus. The antibody is only found in a body liquid, such as blood, and on a cell membrane surface of a B cell of a vertebrate. An antibody level in the blood of a recipient of a novel coronavirus vaccine is usually used to measure the efficacy of the vaccine and whether the recipient has developed protection against the novel coronavirus.

### 4) Antigen (Ag)

An antigen is a substance that can stimulate the body to produce an immune response. It may be foreign matter, such as a virus or a bacterium, a dead protein of the human body or a cell composed of proteins. A foreign molecule can be recognized by an immunoglobulin on a B cell or processed by an antigen-presenting cell, and be combined with a major histocompatibility complex to form a complex that reactivates a T cell and trigger a continuous immune response.

The antibody, as a type of immunoglobulin, is a key component of the immune system. Generally, a complex structure containing a heavy chain and a light chain is referred to as an antibody, while a single-chain structure containing only one peptide chain is referred to as a nanobody (also referred to as a single-domain antibody). A specific antibody can recognize a specific antigen, and specifically bind to the antigen in vivo or in vitro. Prediction of a structure of an antigen-antibody complex helps better understand an action mechanism of the immune system, and can be applied to screening and evaluation of an antibody, which provides strong support for auxiliary diagnosis and auxiliary treatment, and promotes research and development of drugs.

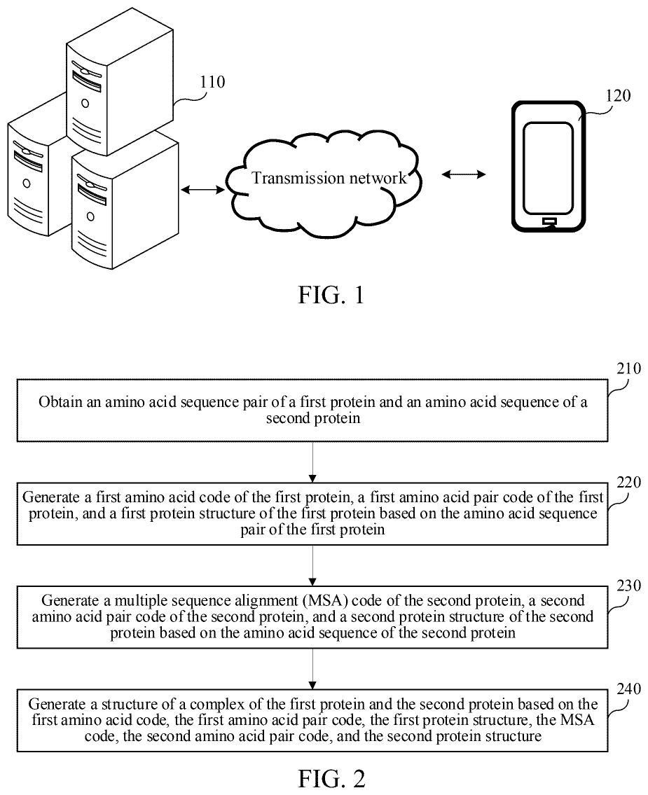

FIG. 1 is a schematic diagram of a system used in a complex structure determination method according to an exemplary embodiment of this application. As shown in FIG. 1, the system includes: a server 110 and a terminal 120.

The server 110 may be an independent physical server, or may be a server cluster or distributed system including a plurality of physical servers, or may be a cloud server providing basic cloud computing services such as a cloud service, a cloud database, cloud computing, a cloud function, cloud storage, a network service, cloud communication, a middleware service, a domain name service, a security service, a content delivery network (CDN), and a big data and AI platform.

The server 110 may include a server that is deployed with a complex structure determination system and provides a complex structure determination service for a user through the complex structure determination system. Alternatively, the server 110 may include a server that is deployed with a complex structure determination system and trains or updates the complex structure determination system.

The terminal 120 may be a terminal device having a network connection function and a data processing function. For example, the terminal 120 is a smartphone, a tablet computer, an e-book reader, smart glasses, a smartwatch, a smart television, a laptop portable computer, a desktop computer, or the like.

The terminal 120 may include a user terminal that accepts a complex structure determination service, or the terminal 120 may include a development terminal used by a developer of a complex structure determination system.

In an embodiment, the terminal 120 is deployed with a complex structure determination system.

In an embodiment, the system includes one or more servers 110, and a plurality of terminals 120. A quantity of servers 110 and a quantity of terminals 120 are not limited in the embodiments of this application.

The terminal and the server are connected over a communication network. In an embodiment, the communication network is a wired network or wireless network.

In an embodiment, the wireless network or wired network uses a standard communication technology and/or protocol. The network is usually the Internet, but may alternatively be any network, including but not limited to, any combination of a local area network (LAN), a metropolitan area network (MAN), a wide area network (WAN), a mobile, wired, or wireless network, a dedicated network, and a virtual dedicated network. In some embodiments, data exchanged over the network are represented in the technology and/or format such as Hypertext Markup Language (HTML) or Extensible Markup Language (XML). All or some links may be further encrypted by a conventional encryption technology such as Secure Socket Layer (SSL), Transport Layer Security (TLS), a virtual private network (VPN), or Internet Protocol Security (IPsec). In some other embodiments, the data communication technologies are replaced or supplemented with a customized and/or dedicated data communication technology. This is not limited in this application.

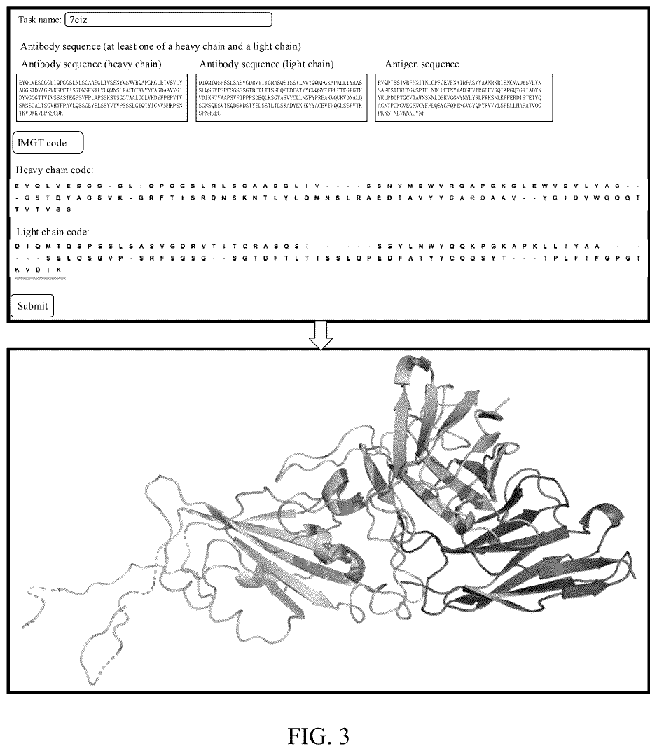

FIG. 2 is a flowchart of a complex structure determination method according to an exemplary embodiment of this application. The method is performed by a computer device. The computer device may be implemented as a terminal or a server. The terminal or the server may be the terminal or the server shown in FIG. 1. As shown in FIG. 2, the complex structure determination method includes the following operations.

Operation 210: Obtain an amino acid sequence pair of a first protein and an amino acid sequence of a second protein.

In the embodiments of this application, the computer device provides an input interface to the external, which is configured to receive the amino acid sequence pair of the first protein and the amino acid sequence of the second protein. The first protein has functions of recognizing the second protein and specifically binding to the second protein.

The second protein may be an antigen, and the first protein may be one antibody corresponding to the antigen.

The antibody is a symmetrical structure with four polypeptide chains, which includes two identical heavy chains (H chains) that are relatively long and have a relatively large relative molecular weight; and two identical light chains (L chains) that are relatively short and have a relatively small relative molecular weight. One heavy chain and one light chain form a pair. The chains are linked by disulfide bonds and a non-covalent bonds to form a monomer molecule containing four polypeptide chains. The entire antibody molecule may be divided into two regions, namely, a constant region and a variable region.

The amino acid sequence pair of the first protein may be a sequence pair composed of respective amino acid sequences of one heavy chain and one light chain that form a pair in one antibody. Due to the symmetrical structure of the antibody, one amino acid sequence pair may represent an amino acid sequence of the entire antibody.

Operation 220: Generate a first amino acid code of the first protein, a first amino acid pair code of the first protein, and a first protein structure of the first protein based on the amino acid sequence pair of the first protein.

In the embodiments of this application, the computer device does not need to perform multiple sequence alignment (MSA) construction on the amino acid sequence of the first protein, but directly performs coding and structure prediction based on the amino acid sequence pair of the first protein, to obtain the first amino acid code of the first protein, the first amino acid pair code of the first protein, and the first protein structure of the first protein.

When the first protein is one antibody corresponding to a particular antigen, because the antigen may correspond to a plurality of different antibodies, if MSA construction is performed on each antibody, as a quantity of antibodies is increased, time and resources required by MSA construction for the plurality of antibodies are also increased. However, according to the solution provided in the embodiments of this application, MAS construction does not need to be performed on the amino acid sequence of the antibody, whereby efficiency of predicting a structure of an antibody-antigen complex is significantly improved.

In a possible implementation, the first protein structure may include position information (such as three-dimensional position coordinates) of each atom in the first protein.

Operation 230: Generate an MSA code of the second protein, a second amino acid pair code of the second protein, and a second protein structure of the second protein based on the amino acid sequence of the second protein.

In the embodiments of this application, for an amino acid sequence of the second protein, the computer device obtains MSA information of the second protein, and then performs subsequent coding and structure prediction based on the MSA information, to obtain the MSA code of the second protein, the second amino acid pair code of the second protein, and the second protein structure of the second protein.

When the second protein is an antigen, although one antigen may correspond to a plurality of different antibodies, a structure of the antigen is unique. When a structure of a complex obtained by binding the antibody to the antigen is predicted, the MSA information of the antigen may be shared between the different antibodies. That is, regardless of the number of types of antibodies, MAS needs to be performed on the antigen only once, which does not significantly affect prediction of structures of complexes obtained by binding different antibodies to the antigen.

In a possible implementation, the second protein structure may include position information (such as three-dimensional position coordinates) of each atom in the second protein.

Operation 240: Generate a structure of a complex of the first protein and the second protein based on the first amino acid code, the first amino acid pair code, the first protein structure, the MSA code, the second amino acid pair code, and the second protein structure.

In the embodiments of this application, the computer device comprehensively predicts, based on execution results of operation 220 and operation 230, the structure of the complex obtained by binding the first protein to the second protein. The complex of the first protein and the second protein may be a substance generated by binding the first protein to the second protein. The complex of the first protein and the second protein is a complex formed by binding an antibody to an antigen, which is also referred to as an immune complex.

For example, first, the computer device performs code fusion based on the first amino acid code, the first amino acid pair code, the MSA code, the second amino acid pair code, the first protein structure, and the second protein structure, to obtain an amino acid feature and an amino acid pair feature of the complex obtained by binding the first protein to the second protein. Then, the computer device performs structure prediction based on the amino acid feature and the amino acid pair feature of the complex, to obtain the structure of the complex. For example, the computer device predicts three-dimensional position coordinates of each atom in the complex based on the amino acid feature and the amino acid pair feature of the complex.

In conclusion, according to the solution provided in the embodiments of this application, first, the respective amino acid codes, amino acid pair codes, and structures of the first protein and the second protein are respectively predicted based on the information about the respective amino acid sequences of the first protein and the second protein. Then, the structure of the complex of the first protein and the second protein is predicted based on the respective amino acid code, amino acid pair code, and structures of the first protein and the second protein. In the solution, MSA construction does not need to be performed on the first protein. In a case that many types of proteins may specifically bind to the second protein, that is, in a case that one antigen corresponds to a plurality of antibodies, a large amount of time required by MSA construction can be saved, whereby the efficiency of predicting the structure of the complex of the first protein and the second protein.

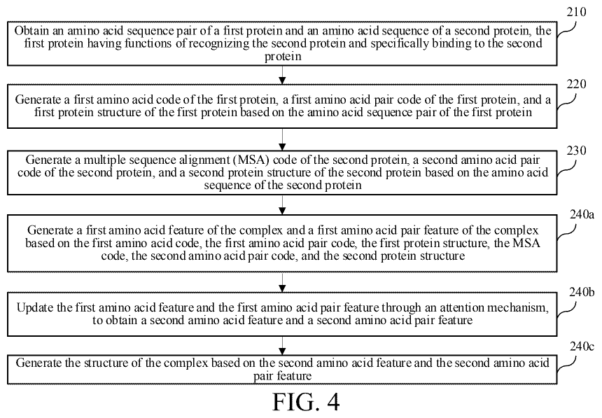

Based on the solution shown in FIG. 2, a flowchart of a complex structure determination method according to an exemplary embodiment of this application is shown in FIG. 4. The method may be performed by a computer device. That is, in the embodiment shown in FIG. 2, operation 240 may be replaced with operation 240a, operation 240b, and operation 240c.

Operation 240a: Generate a first amino acid feature of the complex and a first amino acid pair feature of the complex based on the first amino acid code, the first amino acid pair code, the first protein structure, the MSA code, the second amino acid pair code, and the second protein structure.

In the embodiments of this application, the computer device predicts the structure of the complex by using a complex structure prediction model.

Specifically, the computer device may input the first amino acid code, the first amino acid pair code, the first protein structure, the MSA code, the second amino acid pair code, and the second protein structure into a feature fusion sub-branch in a docking branch of the complex structure prediction model, to obtain the first amino acid feature and the first amino acid pair feature that are outputted by the feature fusion sub-branch.

The process of generating the structure of the complex of the first protein and the second protein based on the first amino acid code, the first amino acid pair code, the first protein structure, the MSA code, the second amino acid pair code, and the second protein structure includes N rounds of prediction. N is an integer greater than or equal to 2.

That is, during prediction of the structure of the complex of the first protein and the second protein, the computer device cyclically performs operation 240 until the N rounds of prediction are completed. In the first N−1 rounds, an output result of each round may be returned as a part of input data of a next round, to implement iterative update of information such as the structure of the complex, the amino acid feature of the complex, and the amino acid pair feature of the complex.

In a possible implementation, the feature fusion sub-branch is configured to respectively extract features of the first amino acid code and the first piece of amino acid code in the MSA code, and concatenate the features to obtain the first amino acid feature; and generate respective amino acid embeddings and amino acid pair embeddings of the first protein and the second protein based on a structure of the complex that is predicted in a previous round, or based on the first protein structure and the second protein structure, and combine the respective amino acid embeddings and amino acid pair embeddings of the first protein and the second protein with the first amino acid code, the first amino acid pair code, a second amino acid code, and the second amino acid pair code, to generate the first amino acid pair feature. The second amino acid code is an amino acid code of the second protein.

In the first round of prediction, because a predicted structure of the complex does not exist, in this case, the computer device may generate the amino acid embedding and the amino acid pair embedding of the first protein based on the first protein structure, and generate the amino acid embedding and the amino acid pair embedding of the second protein based on the second protein structure.

From the second round of prediction to the N^{th} round of prediction, the computer device may generate an amino acid embedding and an amino acid pair embedding of the first protein, as well as an amino acid embedding and an amino acid pair embedding of the second protein in a current round of prediction based on the structure of the complex that is predicted in the previous round. Because the complex is formed by binding the first protein to the second protein, the structure of the complex may be regarded as containing both the structure of the first protein and the structure of the second protein. In this case, by introducing the structure of the complex that is predicted in the previous round into the cyclic prediction process, iterative optimization of the structure of the complex can be implemented, whereby the accuracy of prediction of the structure of the complex is enhanced.

In another possible implementation, from the second round of prediction to the N^{th} round of prediction, the computer device may generate the amino acid embedding and the amino acid pair embedding of the first protein, as well as the amino acid embedding and the amino acid pair embedding of the second protein in the current round of prediction based on the structure of the complex that is predicted in the previous round, as well as the first protein structure and the second protein structure. For example, the computer device splits the structure of the complex that is predicted in the previous round into a structure related to the first protein and a structure related to the second protein, and then adds the structure related to the first protein to the first protein structure (for example, by weighted summation based on a preset weight), to generate the amino acid embedding and the amino acid pair embedding of the first protein; and adds the structure related to the second protein to the second protein structure (for example, by weighted summation based on a preset weight), to generate the amino acid embedding and the amino acid pair embedding of the second protein. According to the solution, not only the structures of the first protein and the second protein may be continuously iterated by cyclical prediction of the structure of the complex, but also an iterative optimization process of the structure of the complex may be restricted based on the structures of the first protein and the second protein that are predicted in the previous operation. In this way, the accuracy of prediction of the structure of the complex is enhanced.

In a possible implementation, the feature fusion sub-branch is configured to respectively extract features of the first amino acid code and the first piece of amino acid code in the MSA code, concatenate the features, and add the concatenated feature to an amino acid feature of the complex that is predicted in the previous round to obtain the first amino acid feature; and generate the respective amino acid embeddings and amino acid pair embeddings of the first protein and the second protein based on the structure of the complex that is predicted in the previous round, or based on the first protein structure and the second protein structure, combine the respective amino acid embeddings and amino acid pair embeddings of the first protein and the second protein with the first amino acid code, the first amino acid pair code, the second amino acid code, and the second amino acid pair code, and add the combination to an amino acid pair feature of the complex that is predicted in the previous round, to generate the first amino acid pair feature.

Specifically, in the first round of the N rounds, the computer device may generate the respective amino acid embeddings and amino acid pair embeddings of the first protein and the second protein based on the first protein structure and the second protein structure, and combine the respective amino acid embeddings and amino acid pair embeddings of the first protein and the second protein with the first amino acid code, the first amino acid pair code, the second amino acid code, and the second amino acid pair code, to generate the first amino acid pair feature. From the second round of the N rounds, the computer device may generate the respective amino acid embeddings and amino acid pair embeddings of the first protein and the second protein based on the structure of the complex that is predicted in the previous round, combine the respective amino acid embeddings and amino acid pair embeddings of the first protein and the second protein with the first amino acid code, the first amino acid pair code, the second amino acid code, and the second amino acid pair code, and add the combination to the amino acid pair feature of the complex that is predicted in the previous round, to generate the first amino acid pair feature.

In the embodiments of this application, in each round of prediction, the feature fusion sub-branch further fuses an amino acid feature and an amino acid pair feature of the complex that are obtained in the previous round of prediction into the current round of prediction, whereby the amino acid feature and the amino acid pair feature of the complex gradually approach accurate values. In this way, the accuracy of prediction of the structure of the complex is enhanced.

Operation 240b: Update the first amino acid feature and the first amino acid pair feature through an attention mechanism, to obtain a second amino acid feature and a second amino acid pair feature.

In a possible implementation, the computer device may input, into an attention sub-branch in the docking branch, the first amino acid feature and the first amino acid pair feature that are outputted by the feature fusion sub-branch, to obtain the second amino acid feature and the second amino acid pair feature that are outputted by the attention sub-branch.

In a possible implementation, the attention sub-branch is configured to update the first amino acid feature through the attention mechanism by taking the first amino acid pair feature as a bias, to obtain the second amino acid feature; and add the first amino acid pair feature to the second amino acid feature, and process the added feature through a triangle update mechanism layer and a conversion layer, to obtain the second amino acid pair feature.

In the embodiments of this application, by taking the first amino acid pair feature as a bias, the first amino acid pair feature and the first amino acid feature are processed, to obtain an updated amino acid feature and an updated amino acid pair feature. The amino acid feature and the amino acid pair feature of the complex are fused, whereby the effect of extracting amino acid-related features is enhanced. In this way, the accuracy of subsequent prediction of the structure of the complex is enhanced.

Operation 240c: Generate the structure of the complex based on the second amino acid feature and the second amino acid pair feature.

In a possible implementation, the computer device may input the second amino acid feature and the second amino acid pair feature into a structure generation sub-branch in the docking branch, to obtain the structure of the complex that is outputted by the structure generation sub-branch.

In a possible implementation, the structure generation sub-branch may be configured to update the second amino acid feature, the second amino acid pair feature, and the structure of the complex that is predicted in the previous round through the attention mechanism; and output a structure of the complex, an amino acid feature of the complex, and an amino acid pair feature of the complex that are predicted in a current round.

In the embodiments of this application, when the structure of the complex is predicted based on the second amino acid feature and the second amino acid pair feature, the amino acid feature and the amino acid pair feature of the complex are further updated through the attention mechanism, to further enhance the effect of fusing the amino acid-related features. In this way, the accuracy of prediction of structure of the complex is enhanced.

The structure of the complex that is outputted in the N^{th} round of prediction is a structure of the complex that is finally outputted by the entire model.

In a possible implementation, operation 220 may be implemented as follows: the computer device inputs the amino acid sequence pair of the first protein into a first structure prediction branch of the complex structure prediction model, to obtain the first amino acid code, the first amino acid pair code, and the first protein structure that are outputted by the first structure prediction branch.

In a possible implementation, operation 230 may be implemented as follows: the computer device inputs the amino acid sequence of the second protein into a second structure prediction branch of the complex structure prediction model, to obtain the MSA code, the second amino acid pair code, and the second protein structure that are outputted by the second structure prediction branch.

In the embodiments of this application, the computer device respectively predicts amino acid-related codes and structures of the two types of proteins by using a machine learning model. In this way, the accuracy of prediction of a protein monomer is enhanced.

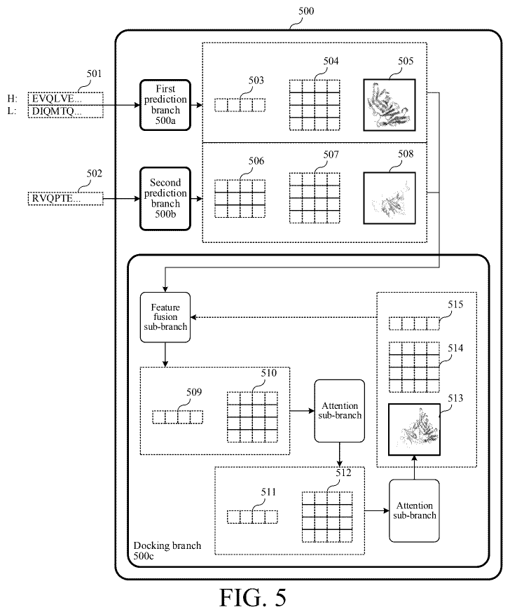

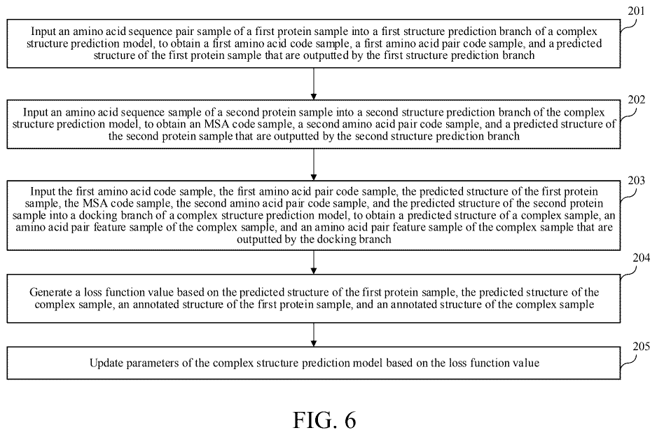

FIG. 5 is a block diagram of a complex structure prediction model according to an embodiment of this application. As shown in FIG. 5, for a first protein and a second protein, a computer device deployed with a complex structure prediction model 500 may perform the following operations to predict a structure of a complex.

S1: The computer device first obtains an amino acid sequence pair 501 of the first protein and an amino acid sequence 502 of the second protein.

In this process, the amino acid sequence pair 501 and the amino acid sequence 502 may be uploaded or entered by a user to the computer device, or pulled from a network/database by the computer device.

S2: The computer device inputs the amino acid sequence pair 501 into a first structure prediction branch 500a of the complex structure prediction model, and inputs the amino acid sequence 502 into a second structure prediction branch 500b of the complex structure prediction model.

The first structure prediction branch 500a outputs a first amino acid code 503, a first amino acid pair code 504, and a first protein structure 505 of the first protein. The second structure prediction branch 500b outputs an MSA code 506, a second amino acid pair code 507, and a second protein structure 508 of the second protein.

S3: The computer device inputs the first amino acid code 503, the first amino acid pair code 504, the first protein structure 505, the MSA code 506, the second amino acid pair code 507, and the second protein structure 508 into a docking branch 500c of the complex structure prediction model, to perform N rounds of prediction on a structure of a complex in the docking branch 500c.

The computer device inputs the first amino acid code 503, the first amino acid pair code 504, the first protein structure 505, the MSA code 506, the second amino acid pair code 507, and the second protein structure 508 into a feature fusion sub-branch in the docking branch 500c, and the feature fusion sub-branch outputs a first amino acid feature 509 and a first amino acid pair feature 510 of the complex.

In addition to the first amino acid code 503, the first amino acid pair code 504, the first protein structure 505, the MSA code 506, the second amino acid pair code 507, and the second protein structure 508, input data of the feature fusion sub-branch further includes a structure of the complex and an amino acid feature and an amino acid pair feature of the complex that are outputted by a structure generation sub-branch in the docking branch 500c in a previous round. In the first round of prediction, the feature fusion sub-branch sets data corresponding to the structure, the amino acid feature, and the amino acid pair feature of the complex that are outputted in the previous round to 0.

S4: In each round of prediction, the computer device inputs a first amino acid feature 509 and a first amino acid pair feature 510 that are outputted by the feature fusion sub-branch in a current round to an attention sub-branch of the complex structure prediction model, and the attention sub-branch outputs a second amino acid feature 511 and a second amino acid pair feature 512 in the current round.

S5: In each round of prediction, the computer device inputs the second amino acid feature 511 and the second amino acid pair feature 512 that are outputted by the attention sub-branch in the current round into a structure generation sub-branch, and the structure generation sub-branch outputs a structure 513 of the complex that is predicted in the current round.

When the N rounds of prediction are completed, the computer device may output the structure of the complex that is predicted by the structure generation sub-branch as a final prediction result. When the N rounds of prediction are not completed, the computer device returns the structure 513 of the complex, an amino acid feature 514 of the complex, and an amino acid pair feature 515 of the complex that are predicted by the structure generation sub-branch to the feature fusion sub-branch for next prediction.

A description is made by using an example in which the first protein and the second protein are respectively an antibody and an antigen, and the model involved in the solution provided in the embodiments of this application is mainly divided into three modules:
1) an antibody structure prediction module (namely, the first structure prediction branch);
2) an antigen structure prediction module (namely, the second structure prediction branch); and
3) an antigen-antibody complex docking module (namely, the docking branch).

In a possible implementation, the antibody structure prediction module takes an amino acid code of a protein language model and an attention matrix as inputs, and may perform iterative update on the amino acid code and an amino acid pair code by using an improved Evoformer network, to obtain amino acid code information and amino acid code pair information of a heavy chain and a light chain, respectively, combine the amino acid code information and the amino acid code pair information of the light chain and the heavy chain of a same antibody, and perform iterative update by using another improved Evoformer network to obtain an amino acid code and an amino acid pair code of an antibody pair. A structure of the antibody is predicted by using a structure generation network based on the updated amino acid code of the antibody and the updated amino acid pair code of the antibody.

The antigen structure prediction module may search a protein sequence library for a homologous sequence based on an antigen sequence, construct MSA, search a protein template library (optional) based on MSA, and input a result into a pre-trained protein structure prediction model to predict a structure of the antigen.

The amino acid code, the amino acid pair code, and the protein structure that are respectively generated by the antigen structure prediction module and the antibody structure prediction module are inputted into the antigen-antibody complex docking module. First, information about an antigen-antibody pair is extracted by using a feature fusion network, to generate an amino acid code of the antigen-antibody complex and an amino acid pair code of the antigen-antibody complex, then, iterative update is performed on the amino acid code and the amino acid pair code of the antigen-antibody complex by using another improved Evoformer network, and finally, a structure of the antigen-antibody complex is predicted by using the structure generation network based on the updated amino acid code of the antigen-antibody complex and the updated amino acid pair code of the antigen-antibody complex.

According to the solution provided in the embodiments of this application, structure prediction is performed based on the amino acid sequence pair of the antibody and MSA of the antigen. For amino acid sequence pairs of different antibodies, time-consuming homologous sequences searching does not need to be performed. This is beneficial to structure-based large-scale screening of antibodies. Meanwhile, according to the method, an entire structure of the complex of the antibody (including a light chain and a heavy chain) and the antigen can be predicted from terminal to terminal, whereby a research and development cycle of an antibody drug is shortened.

The complex structure prediction model is a machine learning module trained based on an amino acid sequence pair sample of a first protein sample, an annotated structure of the first protein sample, an amino acid sequence sample of a second protein sample, and an annotated structure of a complex sample. The complex sample is a product of a specific binding reaction between the first protein sample and the second protein sample.

Based on the solution shown in FIG. 4, a flowchart of a model training method according to an exemplary embodiment of this application is shown in FIG. 6. The method may be performed by a computer device. The method may include the following operations.

Operation 201: Input an amino acid sequence pair sample of a first protein sample into a first structure prediction branch of a complex structure prediction model, to obtain a first amino acid code sample, a first amino acid pair code sample, and a predicted structure of the first protein sample that are outputted by the first structure prediction branch.

Operation 202: Input an amino acid sequence sample of a second protein sample into a second structure prediction branch of the complex structure prediction model, to obtain an MSA code sample, a second amino acid pair code sample, and a predicted structure of the second protein sample that are outputted by the second structure prediction branch.

Operation 203: Input the first amino acid code sample, the first amino acid pair code sample, the predicted structure of the first protein sample, the MSA code sample, the second amino acid pair code sample, and the predicted structure of the second protein sample into a docking branch of the complex structure prediction model, to obtain a predicted structure of a complex sample, an amino acid pair feature sample of the complex sample, and an amino acid pair feature sample of the complex sample that are outputted by the docking branch.

Operation 204: Generate a loss function value based on the predicted structure of the first protein sample, the predicted structure of the complex sample, an annotated structure of the first protein sample, and an annotated structure of the complex sample.

Operation 205: Update parameters of the complex structure prediction model based on the loss function value.

In a possible implementation, the loss function value includes a first loss function value.

The first loss function value includes difference information between the predicted structure of the first protein sample and the annotated structure of the first protein sample.

In a possible implementation, the loss function value includes a second loss function value.

The second loss function value includes difference information between the predicted structure of the complex sample and the annotated structure of the complex sample.

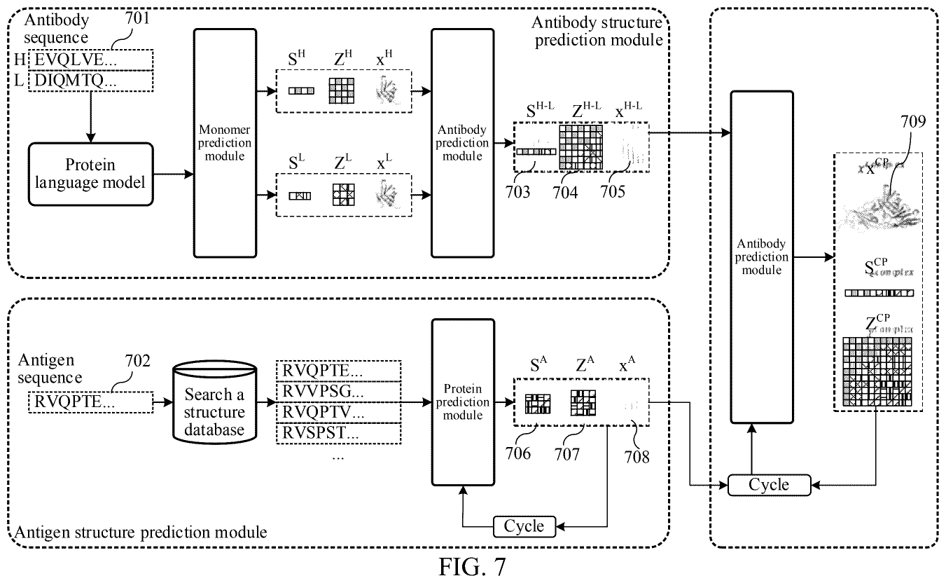

A description is made by using an example in which the first protein and the second protein are respectively an antibody and an antigen, and a structure of a model (which may be referred to as tFold-Ag) involved in the solution provided in the embodiments of this application may be as that shown in FIG. 7.

In FIG. 7, during operation, after a pair of antibody sequences 701 and one antigen sequence 702 are input, an antibody structure prediction module outputs an amino acid code 703 and an amino acid pair code 704 of the antibody, as well as a structure 705 of the antibody, and an antigen structure prediction module outputs an amino acid code 706 (namely, an MSA code) of the antigen, an amino acid pair code 707 of the antigen, and a structure 708 of the antigen. An antigen-antibody complex docking module receives output results of the antibody structure prediction module and the antigen structure prediction module, to output a structure 709 of an antigen-antibody complex.

In FIG. 7, S^{H} is an amino acid code of a heavy chain of the antibody, Z^{H} is an amino acid pair code of the heavy chain of the antibody, and x^{H} is a structure of the heavy chain of the antibody.

S^{L} is an amino acid code of a light chain of the antibody, Z^{L} is an amino acid pair code of the light chain of the antibody, x^{L} is a structure of the light chain of the antibody.

S^{H-L} is the amino acid code of the antigen, Z^{A} is the amino acid pair code of the antigen, x^{A} is the structure of the antigen.

S^{CP} is an amino acid code of the antigen-antibody complex, Z^{CP} is an amino acid pair code of the antigen-antibody complex, and x^{CP} is the structure of the antigen-antibody complex.

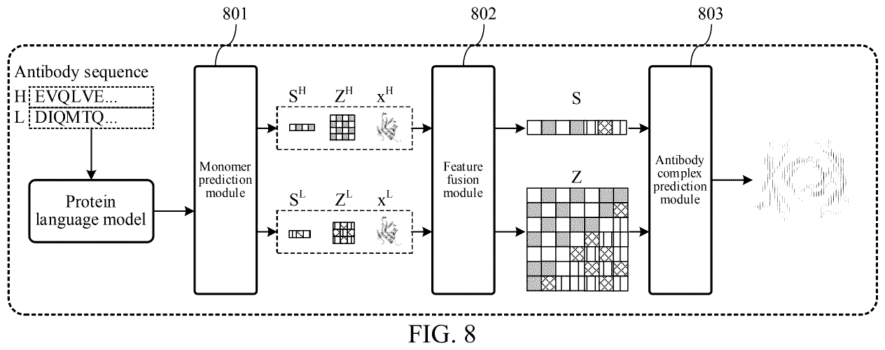

A structure of the antibody structure prediction module (corresponding to the first structure prediction branch) in FIG. 7 may be as that shown in FIG. 8. In FIG. 8, the antibody structure prediction module includes a monomer prediction module 801, a feature fusion module 802, and an antibody complex prediction module 803. The monomer prediction module 801 includes a coding module (integrated with recurrent embedding), an Evoformer-Single module, and a structure generation module.

In a possible implementation, the antibody structure prediction module does not use an iteration policy. Because the antigen-antibody complex docking module can subsequently further optimize a local structure of the antibody, not using the iteration policy can save computational overheads and time.

In FIG. 7, the antigen structure prediction module (corresponding to the second structure prediction branch) may use a pre-trained protein structure prediction model (such as an AlphaFold2 model). The pre-trained protein structure prediction model may be trained based on all proteins in the Protein Data Bank, instead of only based on antigen proteins. Considering the diversity of antigen types and expecting that the model has generalizability and can predict completely novel types of proteins that do not exist in a training set, the pre-trained structure prediction model is used in this application. The antigen structure prediction module may search a protein sequence library by using a tool such as HHblits or Jackhmmer, to generate MAS of antigen sequences. MAS is taken as an input, the antigen structure prediction model outputs not only the structure of the antigen, but also the MSA code and the amino acid pair code of the antigen, as well as a confidence score (pTM) of the predicted structure of the antigen. In the embodiments of this application, difficulty of the structure of the antigen may further be evaluated based on the confidence score of the structure of the antigen. When the pTM score is excessively low, optimization of MSA may be considered (for example, MSA may be optimized by adding a sequence library or changing the search tool), or template information may be added as an additional input. The antigen structure prediction module also supports inputting template information of the antigen. When an actual structure of the antigen exists, in the solution provided in the embodiments of this application, the actual structure of the antigen is also input as a template into the antigen structure prediction module, whereby a better amino acid representation of the antigen and a better amino acid pair representation of the antigen are obtained.

In FIG. 7, the antigen-antibody complex docking module (corresponding to the docking branch) includes a feature fusion module (namely, the feature fusion sub-branch), an Evoformer-Single module (namely, the attention sub-branch), and a structure generation module (namely, the structure generation sub-branch).

The feature fusion module takes the amino acid code of the antibody, the amino acid pair code of the antibody, and the structure of the antibody that are generated by the antibody structure prediction module, as well as the MSA code of the antigen, the amino acid pair code of the antigen, and the structure of the antigen that are generated by the antigen structure prediction module as inputs, to generate an amino acid feature of an antigen-antibody pair (corresponding to the antigen-antibody complex) and an amino acid pair feature of the antigen-antibody pair.

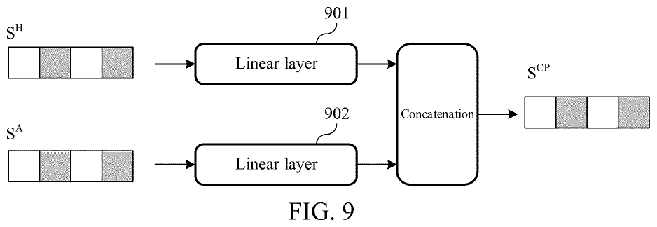

Specifically, a process of generating an amino acid code is shown in FIG. 9. An amino acid code of an antibody sequence is concatenated (for example, by concat) to the first piece of amino acid code (namely, an amino acid representation of an antigen sequence itself) in an MAS code of an antigen sequence through one linear layer 901 and one linear layer 902. From the second round of prediction to the N^{th} round of prediction, the connection result is further added to an amino acid feature of a complex that is predicted in a previous round, to generate an amino acid feature of an antigen-antibody pair.

An amino acid pair code may be implemented by using the OuterProductMean_SM algorithm. That is, amino acid embeddings of monomers (an amino acid embedding of the antibody and an amino acid embedding of the antigen) and amino acid pair embeddings of the monomers (an amino acid pair embedding of the antibody and an amino acid pair embedding of the antigen) are generated based on the structure of the complex that is predicted in the previous round, and the amino acid embeddings of the monomers and the amino acid pair embeddings of the monomers are combined with initial amino acid codes of the monomers (an amino acid code of the antibody and an amino acid code of the antigen that are initially inputted into an antigen-antibody complex docking module) and initial amino acid pair codes of the monomers (an amino acid pair code of the antibody and an amino acid pair code of the antigen that are initially inputted into the antigen-antibody complex docking module), to generate an amino acid feature and an amino acid pair feature that contain information about both the antibody and the antigen. The amino acid feature and the amino acid pair feature that contain the information about both the antibody and the antigen are combined into an amino acid pair feature of the antigen-antibody pair.

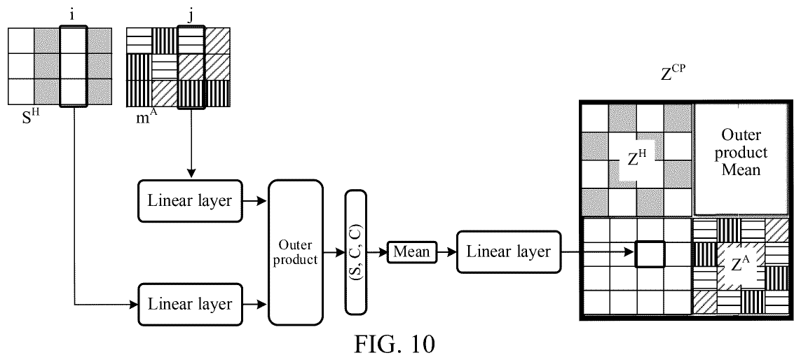

As shown in FIG. 10, m^{A} is an MSA code of an antigen, and a principle of the OuterProductMean_SM algorithm is as follows:

A point (i, j) on the amino acid feature of a complex is equal to a result of an outer product operation of an i^{th} column in an amino acid code of an antibody and a j^{th} column in an amino acid code of the antigen. An operation process of the outer product is as follows: encoded data in the i^{th} column and encoded data in the j^{th} column are respectively expanded to dimensions of [s, 1, c] and [s, c, 1], and then element-wise multiplication is performed. That is, through a broadcast mechanism, the encoded data is expanded to a dimension of [s, c, c], the encoded data with the dimension of [s, c, c] is averaged along the first dimension (mean) to obtain encoded data with a dimension of [1, c, c], the encoded data is reshaped to a dimension of [c*c], and finally, the encoded data is passed through a linear layer to obtain a result at (i, j). s represents a quantity of proteins, and c represents a quantity of amino acid channels.

In other words, an amino acid i may be represented by a matrix of [s, c], and an amino acid j is also represented by the matrix of [s, c]. In the process shown in FIG. 10, two matrices need to be converted into one vector. Both the amino acid i and the amino acid j are passed through the linear layer to be converted to a dimension of [s, c], and then batch dot is performed to obtain encoded data with a dimension of [s, c, c]. The encoded data with the dimension of [s, c, c] is averaged along the s dimension (mean), and a flatten operation (the matrix is converted to a one-dimensional array) is performed to transform the matrix into a vector, and the vector is then passed through the linear layer and added to the amino acid feature of the complex.

Then, the output results in FIG. 9 and FIG. 10 are inputted into Evoformer-Single to update amino acid codes of monomers and amino acid pair codes of the monomers through an attention mechanism, and finally, the updated amino acid codes and the updated amino acid pair codes of the monomers are inputted into a structure generation module to generate a structure of the antigen-antibody complex.

The Evoformer-Single module may include modified versions of 16 to 32 Evoformers, and performs iterative update on the amino acid code and the amino acid pair code. Specifically, the Evoformer module may be set without a column self-attention matrix layer. An inputted amino acid code is updated through a row attention mechanism. In addition to self-attention, an amino acid pair code is further taken as a bias. Then, an updated amino acid code is obtained through a conversion layer. An inputted amino acid pair code is added to the amino acid code that is updated and is subjected to an outer product operation, and then, an updated amino acid pair code is obtained through a triangle update mechanism layer and the conversion layer. The conversion layer sequentially includes a normalization layer, a linear layer, a relu layer, and a linear layer in sequence. The triangle update mechanism layer herein includes an out-triangle product layer, an in-triangle product layer, a triangle attention layer based on initiation site, and a triangle attention layer based on the termination site in sequence.

The structure generation module includes 8 to 16 invariant point attention (IPA) modules sharing a weight. Scalars, points, and attention values in the IPA modules are integrated by addition, to generate an attention map. An amino acid code, an amino acid pair code, and structure information are updated by using the attention map. Finally, the three types of information are added, and are passed through a linear layer to obtain an updated amino acid code. The structure generation module predicts both atomic three-dimensional coordinates (namely, a structure of an antigen-antibody complex) and a confidence score of a monomer structure of the antigen-antibody complex. The confidence score may include pLDDT and pTMscore.

In addition, Evoformer-Single included in the antibody structure prediction module and the antigen structure prediction module is similar to Evoformer-Single in the structure generation module.

The model provided in the embodiments of this application can simultaneously train a single antibody (a nanobody), an antibody, and an antigen-antibody complex structure, and a total target function is as follows:

$${L}_{total} = {w}_{mono} \times ( {L}_{H} + {L}_{L} ) + {w}_{anti} \times {L}_{antibody} + {w}_{comp} \times {L}_{complex}$$

where, L_{H} and L_{L} are target functions of a light chain and a heavy chain, respectively, L_{antibody} is a target function of an antibody, L_{complex} is a target function of an antigen-antibody complex, and wₘₒₙₒ, wₐₙₜᵢ, w_{comp}∈[0,1] are weight coefficients and hyper-parameters. In a training process, if training data is a nanobody, wₘₒₙₒ=1 and wₐₙₜᵢ=0; if the training data is an antibody, wₘₒₙₒ=0.25 and wₐₙₜᵢ=0.5; and if the training data is an antibody-antigen complex, wₘₒₙₒ=0.125, wₐₙₜᵢ=0.25, and w_{comp}=0.5.

Specifically,

$${L}_{H} = {L}_{dist}^{H} + {L}_{rmsd}^{H} + {L}_{fape}^{H} + {L}_{angl}^{H} + {L}_{quat}^{H} + 0.1 \times {L}_{conf}^{H} + 0.1 \times {L}_{viol}^{H}$$

$${L}_{L} = {L}_{dist}^{L} + {L}_{rmsd}^{L} + {L}_{fape}^{L} + {L}_{angl}^{L} + {L}_{quat}^{L} + 0.1 \times {L}_{conf}^{L} + 0.1 \times {L}_{viol}^{L}$$

$${L}_{anti} = {L}_{dist} + {L}_{rmsd} + {L}_{fape} + {L}_{ifape} + {L}_{angl} + {L}_{quat} + 0.1 \times {L}_{conf} + 0.1 \times {L}_{viol}$$

$${L}_{comp} = {L}_{dist} + {L}_{rmsd} + {L}_{fape} + {L}_{ifape} + {L}_{angl} + {L}_{quat} + 0.1 \times {L}_{conf} + 0.1 \times {L}_{viol}$$

where, L_{dist} is a target function for predicting a distance and an included angle between an amino acid pair, L_{rmsd} is a target function for measuring a difference between a predicted structure and an actual structure, L_{fape} is a common frame aligned point error (FAPE) target function (for all atoms in a molecule, an overlap operations is performed on each local frame, a distance is calculated for each atom and an upper limit is set, and summation and averaging are performed to obtain an FAPE), L_{ifape} is an FAPE target function for measuring contact areas between a light chain and a heavy chain, between the light chains, and between the heavy chains, L_{angl} is configured to measure a torsion angle, L_{conf} is configured to measure a confidence score of a light chain, and includes two types of scores, namely, pLDDT and pTMscore, Lᵥᵢₒₗ is configured to evaluate a conflict between amino acids in a predicted structure, and L_{quat} is an auxiliary loss of the structure generation module. Each of the loss functions with a superscript H represents a loss corresponding to a heavy chain, each of the loss functions with a superscript L represents a loss corresponding to a light chain, and each of the loss functions without a superscript represents a loss corresponding to an antibody or an antigen-antibody complex.

The proportion of the different models in the loss function may be adjusted according to an experimental effect.

Hyper-parameters of the foregoing model, such as a quantity of layers of Evoformer-Single and a quantity of layers of the IPA modules, may be adjusted according to a resource requirement.

In the solution provided in the embodiments of this application, a nanobody structure may be trained alone, or an anti-body structure including both a light chain and a heavy chain may be trained alone, or the nanobody structure and the antibody structure may be trained together.

In the solution provided in the embodiments of this application, parameters of the antibody may be fixed, and only the docking module is trained.

In the solution provided in the embodiments of this application, the docking module may perform docking on an antigen and an antibody and may perform docking on common proteins, and may train a sequence and a structure of a common protein, as well as a sequence and a structure of a complex protein.

In the solution provided in the embodiments of this application, Evofomrer may be replaced with another model such as Transformer, AxialTransformer, or ResNet.

The solution provided in the embodiments of this application may be applied to prediction of a structure of any complex generated by specific binding of different proteins.

For example, in a possible application scenario, the solution provided in the embodiments of this application may be applied to prediction of structure of a complex generated by a specific binding reaction between an antibody and an antigen, and may meet a requirement of modeling of structures of antibodies and antigens in the medical industry.

An antibody is a highly specific protein, which can bind to a foreign antigen and produce an immune response. When the antibody interacts with the antigen, chemical bonds of the antibody and the antigen bind to form a plurality of types of antigen-antibody complexes. These complexes play an important role in the immune system, including clearing foreign pathogens and regulating an immune response.

A function of the antibody is determined by the complex formed by interaction between the antibody and the antibody. Understanding of a structure and a function of the antigen-antibody complex is a key to analyzing the function of the antibody. By parsing the structure of the antibody-antigen complex, key information, such as an interaction manner, an action site, and an action strength between the antibody and the antibody can be learned. The information may help scientists better understand the action mechanism of the antibody in an immune response, and precisely improve and optimize the antibody, to enhance recognition and affinity of the antibody for a particular antigen. In addition, understanding of the structure of the antibody-antigen complex may further provide important reference for designing a novel medicine (for example, a treatment drug is developed based on a monoclonal antibody). A potential treatment target is recognized based on the structure of the antibody-antigen complex, and a more effective drug is designed, to accelerate development and launch of a novel drug. In addition, accurate prediction of the structure of the antigen-antibody complex may also play an important role in researching the pathogenesis, diagnosis, and treatment of diseases, to provide better treatment for patients. Therefore, modeling of an antigen-antibody complex is of great value in fields of drag discovery and clinic medicine.

With the development of gene sequencing technology, costs of obtaining an antibody sequence are continuously reduced, and throughput is continuously increased. Usually, tens of thousands of antibody sequences may be obtained by one sampling. However, parsing a three-dimensional structure of an antibody by using an experimental technology is very time-consuming and cost-prohibitive, and parsing a structure of each antibody-antigen complex requires a large amount of time and manpower and material costs. Parsing structures of tens of thousands of antibodies is an inadvisable mode of discovering antibody drugs. Therefore, there is an urgent requirement for fast and low-cost parsing and modeling of a structure of an antibody-antigen complex in the antibody pharmaceutical industry.

In view of the requirement, in the solution provided in this application, a precise and fast AI antibody-antigen complex prediction model is established. As shown in a user interface shown in FIG. 3, a user submits an antibody sequence and an antigen sequence (or MAS or structure of an antigen), and the AI model can complete modeling of a three-dimensional structure of an antibody within few seconds, complete modeling of a three-dimensional structure of an antibody-antigen structure within few minutes, and return a structure file to the user.

After the user enters a heavy chain (or light chain) sequence of the antibody, a backend uniformly recognizes the sequence, determines whether the entered amino acid sequence is an antibody sequence, uniformly encodes the antibody, and divides a complementarity-determining region (CDR) region and a framework region. The user may select whether an antigen sequence needs to be entered. If the antigen sequence is not entered, only a structure of the antibody is generated, and if the antigen sequence is entered, a structure of an antigen-antibody complex is generated. After the user clicks the “submit” below, the backend calls computing power, such as a central processing unit (CPU) or a graphics processing unit, to model the structures of the antibody and the antibody-antigen complex, completes structure prediction within few seconds, and returns the structures to a web page front end shown in FIG. 3 for visual display.

The solution provided in the embodiments of this application has the following advantages.

1) This solution provides an algorithm for predicting a structure of an antibody-antigen complex based on a pre-trained protein language model and a structure prediction model, which can accurately predict a complete structure of an antigen-antigen complex including a side chain within few seconds.

2) This solution provides a new feature fusion method, which can effectively combine information such as a first-order amino acid representation (a first-order MSA representation), a second-order amino acid pair representation, and a third-order predicted structure, to extend prediction of a structure of a monomer to prediction of a structure of a complex. In this solution, a method for predicting a structure of an antigen-antibody complex from terminal to terminal is designed, which can predict structure of an antibody and a nanobody, a structure of an antigen, and a structure of an antigen-antibody complex.

3) Because MSA does not need to be inputted into the antibody module, this solution is faster than a method based on MSA, such as AlphaFold-Multimer.

4) The precision of this solution is relatively high, and especially, compared with another method, the prediction performance for a CDR of an antibody and a relative spatial positions of an antigen and the antibody is better.

In addition to prediction of a structure of an antigen-antibody complex, the solution provided in the embodiments of this application may further be applied to prediction of a structure of a complex of other proteins.

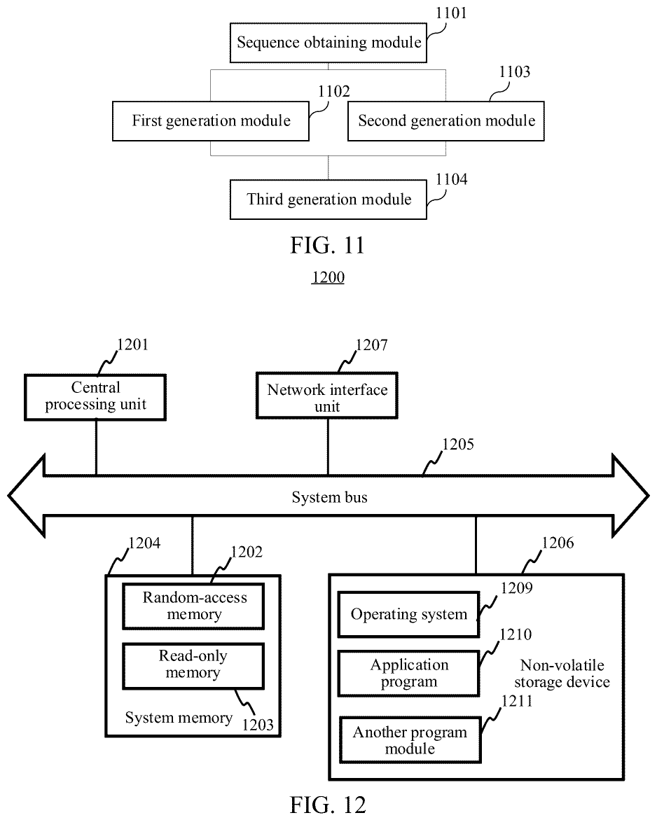

FIG. 11 is a block diagram of a complex structure determination apparatus according to an exemplary embodiment of this application. The apparatus may be configured to perform all or some operations of the method shown in FIG. 2, FIG. 4, or FIG. 6. As shown in FIG. 11, the apparatus includes:

a sequence obtaining module 1101, configured to obtain an amino acid sequence pair of a first protein and an amino acid sequence of a second protein;

a first generation module 1102, configured to generate a first amino acid code of the first protein, a first amino acid pair code of the first protein, and a first protein structure of the first protein based on the amino acid sequence pair of the first protein;

a second generation module 1103, configured to generate an MSA code of the second protein, a second amino acid pair code of the second protein, and a second protein structure of the second protein based on the amino acid sequence of the second protein; and

a third generation module 1104, configured to generate a structure of a complex of the first protein and the second protein based on the first amino acid code, the first amino acid pair code, the first protein structure, the MSA code, the second amino acid pair code, and the second protein structure.

In a possible implementation, the first protein has functions of recognizing the second protein and specifically binding to the second protein.

In a possible implementation, the third generation module 1104 is configured to generate a first amino acid feature of the complex and a first amino acid pair feature of the complex based on the first amino acid code, the first amino acid pair code, the first protein structure, the MSA code, the second amino acid pair code, and the second protein structure; update the first amino acid feature and the first amino acid pair feature through an attention mechanism, to obtain a second amino acid feature and a second amino acid pair feature; and generate the structure of the complex based on the second amino acid feature and the second amino acid pair feature.

In a possible implementation, the third generation module 1104 is configured to input the first amino acid code, the first amino acid pair code, the first protein structure, the MSA code, the second amino acid pair code, and the second protein structure into a feature fusion sub-branch in a docking branch of a complex structure prediction model, to obtain the first amino acid feature and the first amino acid pair feature that are outputted by the feature fusion sub-branch; input, into an attention sub-branch in the docking branch, the first amino acid feature and the first amino acid pair feature that are outputted by the feature fusion sub-branch, to obtain the second amino acid feature and the second amino acid pair feature that are outputted by the attention sub-branch; and input the second amino acid feature and the second amino acid pair feature into a structure generation sub-branch in the docking branch, to obtain the structure of the complex that is outputted by the structure generation sub-branch.

The complex structure prediction model is a machine learning module trained based on an amino acid sequence pair sample of a first protein sample, an annotated structure of the first protein sample, an amino acid sequence sample of a second protein sample, and an annotated structure of a complex sample. The complex sample is a product of a specific binding reaction between the first protein sample and the second protein sample.

In a possible implementation, the process of generating the structure of the complex of the first protein and the second protein based on the first amino acid code, the first amino acid pair code, the first protein structure, the MSA code, the second amino acid pair code, and the second protein structure includes N rounds of prediction. Nis an integer greater than or equal to 2.

The feature fusion sub-branch is configured to respectively extract features of the first amino acid code and the first piece of amino acid code in the MSA code, and concatenate the features to obtain the first amino acid feature; and generate respective amino acid embeddings and amino acid pair embeddings of the first protein and the second protein based on a structure of the complex that is predicted in a previous round, or based on the first protein structure and the second protein structure, and combine the respective amino acid embeddings and amino acid pair embeddings of the first protein and the second protein with the first amino acid code, the first amino acid pair code, a second amino acid code, and the second amino acid pair code, to generate the first amino acid pair feature. The second amino acid code is an amino acid code of the second protein.

In the first round of the N rounds, the feature fusion sub-branch may be configured to generate the respective amino acid embeddings and amino acid pair embeddings of the first protein and the second protein based on the first protein structure and the second protein structure, and combine the respective amino acid embeddings and amino acid pair embeddings of the first protein and the second protein with the first amino acid code, the first amino acid pair code, the second amino acid code, and the second amino acid pair code, to generate the first amino acid pair feature.

From the second round of the N rounds, the feature fusion sub-branch may be configured to generate the respective amino acid embeddings and amino acid pair embeddings of the first protein and the second protein based on the structure of the complex that is predicted in the previous round, and combine the respective amino acid embeddings and amino acid pair embeddings of the first protein and the second protein with the first amino acid code, the first amino acid pair code, the second amino acid code, and the second amino acid pair code, to generate the first amino acid pair feature. The second amino acid code is the amino acid code of the second protein.

In a possible implementation, the attention sub-branch is configured to update the first amino acid feature through the attention mechanism by taking the first amino acid pair feature as a bias, to obtain the second amino acid feature; and add the first amino acid pair feature to the second amino acid feature, and process the added feature through a triangle update mechanism layer and a conversion layer, to obtain the second amino acid pair feature.

In a possible implementation, the structure generation sub-branch is configured to update the second amino acid feature, the second amino acid pair feature, and the structure of the complex that is predicted in the previous round through the attention mechanism; and output a structure of the complex, an amino acid feature of the complex, and an amino acid pair feature of the complex that are predicted in a current round.

In a possible implementation, the feature fusion sub-branch is configured to respectively extract features of the first amino acid code and the first piece of amino acid code in the MSA code, concatenate the features, and add the concatenated feature to an amino acid feature of the complex that is predicted in the previous round to obtain the first amino acid feature; and generate the respective amino acid embeddings and amino acid pair embeddings of the first protein and the second protein based on the structure of the complex that is predicted in the previous round, or based on the first protein structure and the second protein structure, combine the respective amino acid embeddings and amino acid pair embeddings of the first protein and the second protein with the first amino acid code, the first amino acid pair code, the second amino acid code, and the second amino acid pair code, and add the combination to an amino acid pair feature of the complex that is predicted in the previous round, to generate the first amino acid pair feature.

In a possible implementation, the first generation module 1102 is configured to input the amino acid sequence pair of the first protein into a first structure prediction branch of the complex structure prediction model, to obtain the first amino acid code, the first amino acid pair code, and the first protein structure that are outputted by the first structure prediction branch.

The second generation module 1103 is configured to input the amino acid sequence of the second protein into a second structure prediction branch of the complex structure prediction model, to obtain the MSA code, the second amino acid pair code, and the second protein structure that are outputted by the second structure prediction branch.

In a possible implementation, the first generation module 1102 is further configured to input the amino acid sequence pair sample of the first protein sample into the first structure prediction branch of the complex structure prediction model, to obtain a first amino acid code sample, a first amino acid pair code sample, and a predicted structure of the first protein sample that are outputted by the first structure prediction branch.

The second generation module 1103 is further configured to input the amino acid sequence sample of the second protein sample into the second structure prediction branch of the complex structure prediction model, to obtain an MSA code sample, a second amino acid pair code sample, and a predicted structure of the second protein sample that are outputted by the second structure prediction branch.

The third generation module 1104 is further configured to input the first amino acid code sample, the first amino acid pair code sample, the predicted structure of the first protein sample, the MSA code sample, the second amino acid pair code sample, and the predicted structure of the second protein sample into the docking branch of the complex structure prediction model, to obtain a predicted structure of the complex sample that is outputted by the docking branch.

The apparatus further includes:
a loss function value generating module, configured to generate a loss function value based on the predicted structure of the first protein sample, the predicted structure of the complex sample, the annotated structure of the first protein sample, and the annotated structure of the complex sample; and
a parameter updating module, configured to update parameters of the complex structure prediction model based on the loss function value.

In a possible implementation, the loss function value includes a first loss function value.

The first loss function value includes difference information between the predicted structure of the first protein sample and the annotated structure of the first protein sample.

In a possible implementation, the loss function value includes a second loss function value.

The second loss function value includes difference information between the predicted structure of the complex sample and the annotated structure of the complex sample.

FIG. 12 is a structural block diagram of a computer device 1200 according to an exemplary embodiment of this application. The computer device may be implemented as a server or a terminal in the solution of this application. In this embodiment, a description is made by using an example in which the computer device is a server. The computer device 1200 includes a CPU 1201, a system memory 1204 including a random-access memory (RAM) 1202 and a read-only memory (ROM) 1203, and a system bus 1205 connecting the system memory 1204 to the CPU 1201. The computer device 1200 further includes a non-volatile storage device 1206 configured to store an operating system 1209, an application program 1210, and another program module 1211.

The non-volatile storage device 1206 is connected to the CPU 1201 through a mass storage controller (not shown) connected to the system bus 1205. The non-volatile storage device 1206 and a computer-readable medium associated with the non-volatile storage device provide non-volatile storage for the computer device 1200. That is, the non-volatile storage device 1206 may include a computer-readable medium (not shown) such as a hard disk or a compact disc ROM (CD-ROM) drive.

Generally, the computer-readable medium may include a computer storage medium and a communication medium. The computer storage medium includes volatile and non-volatile media, and removable and non-removable media that are implemented by any method or technology for storing information such as computer-readable instructions, data structures, program modules, or other data. The computer storage medium includes an RAM, an ROM, an erasable programmable ROM (EPROM), an electrically EPROM (EEPROM), a flash memory or another solid-state storage technology, a CD-ROM, a digital versatile disc (DVD) or another optical storage, a cassette, a magnetic tape, a disk storage, or another magnetic storage device. Certainly, those skilled in art may understand that the computer storage medium is not limited to the above several types. The system memory 1204 and the non-volatile storage device 1206 may be collectively referred to as a memory.

According to the embodiments of this application, the computer device 1200 may further be connected, over a network such as the Internet, to a remote computer on the network and run. That is, the computer device 1200 may be connected to the network through a network interface unit 1207 connected to the system bus 1205, or may be connected to another type of network or a remote computer system (not shown) through the network interface unit 1207.

The memory further includes at least one computer program. The at least one computer program is stored in the memory. The CPU 1201 executes the at least one computer program to implement all or some operations of the method provided in the embodiments.

In an exemplary embodiment, a computer-readable storage medium is further provided, which is configured to store at least one computer program. A processor loads and executes the computer program to implement all or some operations of the method provided in the embodiments. For example, the computer-readable storage medium is an ROM, an RAM, a CD-ROM, a magnetic tape, a floppy disk, or an optical data storage device.

In another exemplary embodiment, a computer program product is further provided, which includes a computer program. The computer program is stored in a computer-readable storage medium. A processor of a computer device reads the computer program from the computer-readable storage medium, and executes the computer program, to cause the computer device to perform all or some operations of the method provided in the embodiments.

After considering the description and practicing this application, those skilled in the art may easily conceive of other implementations of this application. This application is intended to cover any variation, use, or adaptive change of this application. These variations, uses, or adaptive changes follow the general principles of this application and include common general knowledge or conventional technical means in the art, which are not disclosed in this application. The description and the embodiments are considered as merely exemplary, and the scope and spirit of this application are pointed out in the following claims.

This application is not limited to the precise structures described above and shown in the accompanying drawings, and various modifications and changes may be made without departing from the scope of this application.

The technical features of the embodiments may be combined in different manner to form other embodiments. To make description concise, not all possible combinations of the technical features in the embodiments are described. However, the combinations of these technical features are considered as falling within the scope recorded by the description provided that no conflict exists.

The embodiments only describe several implementations of this application, which are described specifically and in detail, but cannot be construed as a limitation to the patent scope of this application. For those of ordinary skill in the art, several transformations and improvements may be made without departing from the idea of this application. These transformations and improvements fall within the scope of protection of this application. Therefore, the scope of protection of this application is subject to the appended claims.

What is claimed is:

1. A determination method, performed by a computer device, comprising:
obtaining an amino acid sequence pair of a first protein and an amino acid sequence of a second protein;
generating an amino acid code of the first protein, a first amino acid pair code of the first protein, and a first protein structure of the first protein based on the amino acid sequence pair of the first protein;
generating a multiple sequence alignment (MSA) code of the second protein, a second amino acid pair code of the second protein, and a second protein structure of the second protein based on the amino acid sequence of the second protein; and
generating a structure of a complex of the first protein and the second protein based on the amino acid code, the first amino acid pair code, the first protein structure, the MSA code, the second amino acid pair code, and the second protein structure.

2. The method according to claim 1, wherein generating the structure of the complex includes:
generating a first amino acid feature of the complex and a first amino acid pair feature of the complex based on the amino acid code, the first amino acid pair code, the first protein structure, the MSA code, the second amino acid pair code, and the second protein structure;
updating the first amino acid feature and the first amino acid pair feature through an attention mechanism, to obtain a second amino acid feature and a second amino acid pair feature; and
generating the structure of the complex based on the second amino acid feature and the second amino acid pair feature.

3. The method according to claim 2, wherein generating the first amino acid feature and the first amino acid pair feature includes:
inputting the amino acid code, the first amino acid pair code, the first protein structure, the MSA code, the second amino acid pair code, and the second protein structure into a feature fusion sub-branch in a docking branch of a complex structure prediction model, to obtain the first amino acid feature and the first amino acid pair feature.

4. The method according to claim 3, wherein:
the amino acid code is a first amino acid code;
generating the structure of the complex includes N rounds of prediction, N being an integer greater than or equal to 2; and
inputting the amino acid code, the first amino acid pair code, the first protein structure, the MSA code, the second amino acid pair code, and the second protein structure into the feature fusion sub-branch to obtain the first amino acid feature and the first amino acid pair feature includes:
inputting the first amino acid code, the first amino acid pair code, the first protein structure, the MSA code, the second amino acid pair code, and the second protein structure into the feature fusion sub-branch; and
generating, through the feature fusion sub-branch, the first amino acid feature and the first amino acid pair feature, including:
extracting a feature of the first amino acid code and a feature of the first piece of amino acid code in the MSA code, and concatenating the feature of the first amino acid code and the feature of the first piece of amino acid code in the MSA code to obtain the first amino acid feature; and
in any round of the N rounds:
generating first amino acid embedding and first amino acid pair embedding of the first protein and second amino acid embedding and second amino acid pair embedding of the second protein; and
combining the first amino acid embedding, the first amino acid pair embedding, the second amino acid embedding, and the second amino acid pair embedding with the first amino acid code, the first amino acid pair code, a second amino acid code of the second protein, and the second amino acid pair code, to generate the first amino acid pair feature;
wherein:
in the first round of the N rounds, the first amino acid embedding, the first amino acid pair embedding, the second amino acid embedding, and the second amino acid pair embedding are generated based on the first protein structure and the second protein structure; and
in one of the N rounds other than the first round, the first amino acid embedding, the first amino acid pair embedding, the second amino acid embedding, and the second amino acid pair embedding are generated based on the structure of the complex that is predicted in a previous round.

5. The method according to claim 4, wherein extracting the feature of the first amino acid code and the feature of the first piece of amino acid code and concatenating the feature of the first amino acid code and the feature of the first piece of amino acid code to obtain the first amino acid feature includes:
extracting the feature of the first amino acid code and the feature of the first piece of amino acid code, concatenating the feature of the first amino acid code and the feature of the first piece of amino acid code to obtain a concatenated feature, and adding the concatenated feature to an amino acid feature of the complex that is predicted in a previous round to obtain the first amino acid feature.

6. The method according to claim 4, wherein combining the first amino acid embedding, the first amino acid pair embedding, the second amino acid embedding, and the second amino acid pair embedding with the first amino acid code, the first amino acid pair code, the second amino acid code, and the second amino acid pair code, to generate the first amino acid pair feature includes:
combining the first amino acid embedding, the first amino acid pair embedding, the second amino acid embedding, and the second amino acid pair embedding with the first amino acid code, the first amino acid pair code, the second amino acid code, and the second amino acid pair code to obtain a combination, and adding the combination to an amino acid pair feature of the complex that is predicted in the previous round, to generate the first amino acid pair feature.

7. The method according to claim 2, wherein updating the first amino acid feature and the first amino acid pair feature through the attention mechanism includes:
inputting the first amino acid feature and the first amino acid pair feature into an attention sub-branch in a docking branch of a complex structure prediction model, to obtain the second amino acid feature and the second amino acid pair feature.

8. The method according to claim 7, wherein inputting the first amino acid feature and the first amino acid pair feature into the attention sub-branch to obtain the second amino acid feature and the second amino acid pair feature includes:
inputting the first amino acid feature and the first amino acid pair feature to the attention sub-branch; and
in the attention sub-branch:
updating the first amino acid feature through the attention mechanism using the first amino acid pair feature as a bias, to obtain the second amino acid feature; and
adding the first amino acid pair feature to the second amino acid feature, and processing through a triangle update mechanism layer and a conversion layer, to obtain the second amino acid pair feature.

9. The method according to claim 2, wherein generating the structure of the complex based on the second amino acid feature and the second amino acid pair feature includes:
inputting the second amino acid feature and the second amino acid pair feature into a structure generation sub-branch in a docking branch of a complex structure prediction model, to obtain the structure of the complex.

10. The method according to claim 9, wherein:
generating the structure of the complex includes N rounds of prediction, N being an integer greater than or equal to 2; and
inputting the second amino acid feature and the second amino acid pair feature into the structure generation sub-branch to obtain the structure of the complex includes:
inputting the second amino acid feature and the second amino acid pair feature into the structure generation sub-branch; and
updating, using the structure generation sub-branch and through the attention mechanism, the second amino acid feature, the second amino acid pair feature, and the structure of the complex that is predicted in a previous round to generate a structure of the complex, an amino acid feature of the complex, and an amino acid pair feature of the complex as a prediction in a current round.

11. The method according to claim 1, wherein generating the amino acid code, the first amino acid pair code, and the first protein structure includes:
inputting the amino acid sequence pair of the first protein into a structure prediction branch of a complex structure prediction model, to obtain the amino acid code, the first amino acid pair code, and the first protein structure.

12. The method according to claim 1, wherein generating the MSA code, the second amino acid pair code, and the second protein structure includes:
inputting the amino acid sequence of the second protein into a structure prediction branch of a complex structure prediction model, to obtain the MSA code, the second amino acid pair code, and the second protein structure.

13. The method according to claim 1, further comprising:
training a complex structure prediction model for use in generation of the structure of the complex, including:
training the complex structure prediction model based on an amino acid sequence pair sample of a first protein sample, an annotated structure of the first protein sample, an amino acid sequence sample of a second protein sample, and an annotated structure of a complex sample, the complex sample being a product of a specific binding reaction between the first protein sample and the second protein sample.

14. The method according to claim 13, wherein training the complex structure prediction model includes:
inputting the amino acid sequence pair sample of the first protein sample into a first structure prediction branch of the complex structure prediction model, to obtain an amino acid code sample, a first amino acid pair code sample, and a predicted structure of the first protein sample;
inputting the amino acid sequence sample of the second protein sample into a second structure prediction branch of the complex structure prediction model, to obtain an MSA code sample, a second amino acid pair code sample, and a predicted structure of the second protein sample;
inputting the amino acid code sample, the first amino acid pair code sample, the predicted structure of the first protein sample, the MSA code sample, the second amino acid pair code sample, and the predicted structure of the second protein sample into a docking branch of the complex structure prediction model, to obtain a predicted structure of the complex sample;
generating a loss function value based on the predicted structure of the first protein sample, the predicted structure of the complex sample, the annotated structure of the first protein sample, and the annotated structure of the complex sample; and
updating parameters of the complex structure prediction model based on the loss function value.

15. The method according to claim 14, wherein the loss function value includes difference information between the predicted structure of the first protein sample and the annotated structure of the first protein sample.

16. The method according to claim 14, wherein the loss function value includes difference information between the predicted structure of the complex sample and the annotated structure of the complex sample.

17. A computer device comprising:
a processor; and
a memory storing at least one computer program that, when executed by the processor, causes the processor to:
obtain an amino acid sequence pair of a first protein and an amino acid sequence of a second protein;
generate an amino acid code of the first protein, a first amino acid pair code of the first protein, and a first protein structure of the first protein based on the amino acid sequence pair of the first protein;
generate a multiple sequence alignment (MSA) code of the second protein, a second amino acid pair code of the second protein, and a second protein structure of the second protein based on the amino acid sequence of the second protein; and
generate a structure of a complex of the first protein and the second protein based on the amino acid code, the first amino acid pair code, the first protein structure, the MSA code, the second amino acid pair code, and the second protein structure.

18. The computer device according to claim 17, wherein the at least one computer program, when executed by the processor, further causes the processor to, when generating the structure of the complex:
generate a first amino acid feature of the complex and a first amino acid pair feature of the complex based on the amino acid code, the first amino acid pair code, the first protein structure, the MSA code, the second amino acid pair code, and the second protein structure;
update the first amino acid feature and the first amino acid pair feature through an attention mechanism, to obtain a second amino acid feature and a second amino acid pair feature; and
generate the structure of the complex based on the second amino acid feature and the second amino acid pair feature.

19. The computer device according to claim 18, wherein the at least one computer program, when executed by the processor, further causes the processor to, when generating the first amino acid feature and the first amino acid pair feature:
input the amino acid code, the first amino acid pair code, the first protein structure, the MSA code, the second amino acid pair code, and the second protein structure into a feature fusion sub-branch in a docking branch of a complex structure prediction model, to obtain the first amino acid feature and the first amino acid pair feature.

20. A non-transitory computer-readable storage medium storing at least one computer program that, when executed by a processor, causes the processor to:
obtain an amino acid sequence pair of a first protein and an amino acid sequence of a second protein;
generate an amino acid code of the first protein, a first amino acid pair code of the first protein, and a first protein structure of the first protein based on the amino acid sequence pair of the first protein;
generate a multiple sequence alignment (MSA) code of the second protein, a second amino acid pair code of the second protein, and a second protein structure of the second protein based on the amino acid sequence of the second protein; and
generate a structure of a complex of the first protein and the second protein based on the amino acid code, the first amino acid pair code, the first protein structure, the MSA code, the second amino acid pair code, and the second protein structure.
